(19) United States
(12) Patent Application Publication
(10) Pub. No.: US 2015/0361013 A1
(43) Pub. Date: Dec. 17, 2015

(54) USE OF ALIPHATIC NITROSO COMPOUNDS AS INHIBITORS OF RADICAL POLYMERIZATION OF ACTIVATED VINYL MONOMERS

(71) Applicant: GENERAL ELECTRIC COMPANY, Schenectady, NY (US)

(72) Inventors: Tamaz GULIASHVILI, Trevose, PA (US); Ali FADHEL, The Woodlands, TX (US)

(73) Assignee: GENERAL ELECTRIC COMPANY, Schenectady, NY (US)

(21) Appl. No.: 14/764,286

(22) PCT Filed: Feb. 19, 2013
(86) PCT No.: PCT/US13/26658

§ 371 Date: Jul. 29, 2015

(51) Int. Cl. C07C 7/20

(52) U.S. Cl. CPC C07C 7/20

(57) ABSTRACT

Methods and compositions are provided for inhibiting the polymerization of a vinyl aromatic monomer, such as styrene monomer, during elevated temperature processing thereof or during storage or shipment of polymer containing product. The methods comprise adding from about 1-10,000 parts of an aliphatic nitroso compound (ANC) to the monomer, based upon one million parts of the monomer. The compositions comprise a combination of ANC (I) and dinitrophenol (II) and/or quinone methide (III) dissolved or dispersed in a liquid carrier.

### FIELD OF THE INVENTION

Methods and compositions are provided for inhibiting the polymerization of activated vinyl monomers, such as styrene monomer during storage, shipment, or processing. The methods comprise adding from about 1-10,000 parts of an aliphatic nitroso compound polymerization inhibitor to the monomer containing medium, per one million parts of the monomer (parts per million, ppm).

### BACKGROUND OF THE INVENTION

Vinyl monomers are commonly polymerized to form a variety of useful polymer products. For example, styrene, acrylic acid, methacrylic acid, acrylates, methacrylates, and acrylamides are all desirable polymer products formed from controlled polymerization of the vinyl containing monomers corresponding thereto. Other vinyl monomers that are commonly used in polymerization processes include vinyl chloride, vinyl fluoride. and vinyl acetate. When the monomeric vinyl groups are “activated” by an adjacent electron withdrawing group, the increased polarization of the ethenyl bond can give rise to uncontrolled polymerization of the monomer. Styrene, an aromatic vinyl monomer, is an example of an activated vinyl monomer that can undesirably polymerize readily when heated or exposed to light.

Polystyrene is a thermoplastic with many desirable characteristics. It is clear, transparent, readily colored, and easily fabricated. The family of styrene polymers includes polystyrene itself, copolymers of styrene with other vinyl monomers, polymers of derivatives of styrene and mixtures of polystyrene and styrene-containing copolymers with elastomers.

ABS (acrylonitrile, butadiene-styrene) resins have enjoyed tremendous commercial popularity for many years as durable, temperature, and solvent resistant elastomers. On the other hand, styrene plastics are commonly used for packaging, including foams and films, coatings, in appliance fabrication, for housewares and toys, lighting fixtures, and in construction materials.

It is well known that styrene monomer readily polymerizes when heated or exposed to light. In fact, polymerization increases exponentially with increasing temperatures. This polymerization is undesirable during many stages of the manufacturing, processing, separation, handling, storage, and use of styrene monomers.

Common industrial methods for producing styrene include a variety of purification processes, including distillation, to remove impurities. Unfortunately, purification operations carried out at elevated temperatures result in an increased rate of undesired polymerization. Polymerization, such as thermal polymerization, during the monomer purification process, results not only in loss of desired monomer end-product, but also in loss of production efficiency caused by polymer formation or build-up on process equipment. In heat transfer equipment, buildup of polymer adversely affects heat transfer efficiency and may require shut downs of production.

### SUMMARY OF THE INVENTION

In accordance with one aspect of the invention, a method for inhibiting the polymerization of an activated vinyl monomer is provided. The monomer is contacted with an effective polymerization inhibiting amount of an aliphatic nitroso compound (ANC). In other exemplary embodiments, the activated vinyl monomer is a member or members chosen from the group consisting of styrene, acrylic acid, acrylates, methacrylic acid, methacrylates, acrylamides, vinyl chloride, vinyl fluoride, and vinyl acetate. In certain specific embodiments, the activated vinyl monomer is styrene.

In other embodiments, the activated vinyl monomer, in addition to being contacted by an aliphatic nitroso compound, is also contacted by a dinitrophenol compound and/or a quinone methide. In other embodiments, the activated vinyl monomer is processed at elevated temperatures of 80° C. or higher, and polymerization of the monomer is inhibited during such high temperature treatment processing. In other embodiments, the activated vinyl monomer is processed at elevated temperatures of 95° C. or higher.

In certain embodiments the aliphatic nitroso compound may comprise 2 nitroso-2 methylpropane or, in some instances, 2-nitroso-2,4,4-trimethylpentane. The dinitrophenol compound may, for example, comprise 4,6-dinitro-o-cresol, 2,6-dinitro-p-cresol or 2-sec-butyl-4,6-dinitrophenol.

In other specific embodiments, the quinone methide is 2,6-di-tertbutyl-4-benzylidene-cyclohexa-2,5 dien-1-one.

In some aspects of the invention, compositions are provided to inhibit polymerization of activated vinyl monomers. These compositions may comprise an aliphatic nitroso compound in combination with a dinitrophenol and/or a quinone methide. In certain embodiments, the weight ratio in the composition is from about 1 part of the ANC to about 1-5 parts of the combination of dinitrophenol and/or quinone methide.

### DETAILED DESCRIPTION OF EXEMPLARY EMBODIMENTS

In one aspect of the invention, an aliphatic nitroso compound (ANC) is brought into contact with an activated vinyl monomer in order to inhibit polymerization of that monomer. As used throughout, the phrase “activated vinyl monomer” is used to denote a vinyl monomer of the formula CH₂═CH(Me)-X, wherein X is an electron withdrawing group such as phenyl, ester, amide, etc. Exemplary activated monomers include styrene as well as acrylic acid, (meth)acrylic acid, acrylates, (meth)acrylates, acrylamides, vinyl halides, such as vinyl chloride and vinyl chloride, and vinyl acetate.

As to the ANCs that may be useful to inhibit polymerization of activated vinyl monomers, these may be represented by general formula (I)

R—N═O (I)

wherein R represents an unsubstituted or substituted alkyl group, preferably a C₁-C₁₀ alkyl group. In certain embodiments, R is a tertiary alkyl group (e.g., tertiary butyl or octyl).

Exemplary ANCS include the tertiary alkyl nitroso compounds such as 2-nitroso-2-methylpropane and 2-nitroso-2,4,4-trimethylpentane. These ANCs are commercially available and may be prepared via oxidation of the corresponding amines with H₂O₂.

Although applicants are not to be bound to any particular theory of operation, it is thought that the ANC reacts with free radicals generated during heat treatment processing of the activated vinyl monomer, forming so-called oligomeric nitroxide compounds that trap another free radical, thus inhibiting monomer polymerization.

The ANC (I) may he conjointly employed along with (II) a dinitrophenol compound or a (III) quinone methide (QM) compound or both. As to the dinitrophenol compound that may be used, these are represented by the following general Formula (II)

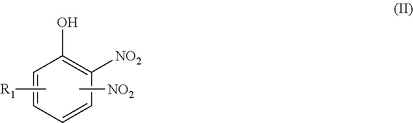

wherein R₁ is H or C₁-C₁₂ alkyl. In certain embodiments, the dinitrophenol compounds include 4,6-dinitro-o-cresol (DNOC), 2,6-dinitro-p-cresol (DNPC) and 2-sec-butyl-4,6,-dinitrophenol (DNBP).

As to the QM compounds that may be conjointly used with the ANC compound in accordance with certain aspects of the invention, these may be seen in the general formula III:

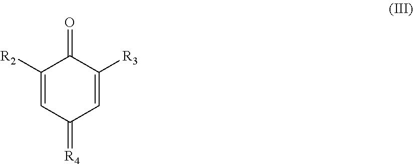

wherein R₂ and R₃ are independently H, C₁ to C₁₈ alkyl, C₅ to C₁₂ cycloalkyl; or C₇ to C₁₅ phenylalkyl, and R₄ is aryl, or aryl substituted with C₁ to C₆ alkyl, alkoxy, hydroxy, nitro, amino, carboxy or mixtures thereof. Means for preparing these quinone methide compounds may be found in U.S. Pat. No. 4,032,547, the contents of which are wholly incorporated by reference herein. Preferably, the quinone methide derivative is 2,6-di-tert-butyl-4-benzylidene-cyclohexa-2,5-dienone.

Preliminary indications demonstrate that the invention is particularly useful to inhibit polymerization of a vinyl aromatic monomer, namely styrene, undergoing distillation and purification processes at elevated temperatures. Styrene, for example, is typically processed at temperatures of between about 95° C.-125° C. The methods, in one aspect of the invention, provide particular efficacy in higher temperature (i.e., >110° C.) styrene processing systems.

The total amount of ANC compound used in the methods is that amount which is sufficient to inhibit polymerization and will vary according to the conditions under which the activated vinyl monomer is being processed and subjected to higher temperatures.

Preferably, the amount of polymerization inhibitor, ANC (I), or combined amount of (I) ANC and (II) DNP and/or (III) QM that is added to the activated vinyl monomer is from about 1 to about 10,000 parts per million parts of monomer. More preferably, the amount is in the range of about 500 parts-1,000 parts per million parts of the monomer. When a combination of ANC/DNP or ANC/QM is used, the weight ratio of ANC/DNP and/or QM is from about 10-1 parts ANC (I) to about 1-10 parts of the combination of DNP (II) and/or QM (III). In some aspects of the invention, this ratio may be about 1 part ANC (I) to about 1-5 parts of the combination of DNP (II) and/or QM (III).

The polymerization inhibitor can be added to the activated vinyl monomer by any conventional method. The polymerization inhibitor may be added to the desired monomer as either a dispersion or as a solution using a suitable liquid carrier or solvent. Any solvent that is compatible with the polymerization inhibitor and the activated vinyl monomer may be employed. Suitable liquid carriers include non-polar organic solvents such as heavy aromatic naptha and xylene.

The efficacy of the polymerization inhibitor treatments was evaluated by the following procedure.

### Procedure

1. Remove the TBC inhibitor by passing reagent grade styrene through a column packed with aluminum oxide. Only use enough for one day's worth of experiment. Do not keep overnight.

2. Prepare filter papers by washing in methanol and drying in a 100° C. oven. Cool filter papers in a dessicator.

3. Place a test-tube rack into a recirculating hot oil bath and heat to 120° C.

4. To an empty 40 mL test tube, add 20 mL of uninhibited styrene and dose with an inhibitor.

5. Close the test tube(s) with a septum and bubble argon gas through the styrene at room temperature for 3-5 minutes. Be sure to vent the tube with another needle while purging.

6. Place the purged tube into the 120° C. bath

7. With a syringe and needle, thief 2.5 mL samples at thirty minute intervals into separate 50 mL beakers. Do this for 4 hours.

8. Precipitate the polystyrene by adding 47.5 mL of methanol to each 2.5 mL sample of styrene.

9. Record the tare weight of a prepared filter paper, T_{F}.

10. Filter the precipitated polystyrene through the tared filter paper.

11. Weigh the polymer and the filter paper together and record the mass.

12. Calculate the mass of polymer and % polymer at each sample interval using the equation below.

### Calculations

$$Mass of polymer \text{:} {M}_{P} = {M}_{F} - {T}_{F}$$

$$\% Polymer \text{:} \% P = \frac{{M}_{P}}{22.75}$$

Styrene Polymerization Results are shown in Table I.

TABLE 1
Temperature 120° C.
Duration: 4 hours

| | | | | QM-1 | DNBP | | |
|---|---|---|---|---|---|---|---|
| | Time in | | MNPD | M̅N̅P̅D̅ | M̅D̅P̅D̅ | QM-1 | DNBP |
| | Minutes | ppm: | 600 | 450/150 | 450/150 | 600 | 600 |
| Mg | 0 | | 0 | 0 | 0 | 0 | 0 |
| 2.5 ml | 60 | | 3 | 3.3 | 5.3 | 7.7 | 7.1 |
| | 120 | | 12.2 | 9.7 | 16.5 | 20.7 | 11.8 |
| | 180 | | 16.1 | 21.5 | 31.8 | 35.6 | 40.8 |
| | 240 | | 301.8 | 35.3 | 64.2 | 55.8 | 61 |
| % | 0 | | 0 | 0 | 0 | 0 | 0 |
| polymer | 60 | | 0.13 | 0.15 | 0.23 | 0.34 | 0.31 |
| | 120 | | 0.54 | 0.43 | 0.73 | 0.91. | 0.52 |
| | 180 | | 0.71 | 0.95 | 1.40 | 1.56 | 1.79 |
| | 240 | | 13.27 | 1.55 | 2.82 | 2.45 | 2.68 |

MNPD = 2-methyl-2-nitroso propane
QM-1 = 4-benzylidene-2,6-ditert-butyl-2,5-cyclohexadien-1-one
DNBP = 2-sec-butyl-4,6-dinitrophenol

Activated vinyl monomer polymerization inhibition compositions in accordance with certain exemplary embodiments of the invention comprise, in combination, an (I) ANC and a (II) DNP and/or a (III) QM. The (I) ANC, in such compositions, is present in an amount by weight of about 1 part to about 1-5 parts of the combination of (II) DNP and/or (III) QM. The combination may be provided as either a dispersion or solution using a suitable liquid solvent or liquid carrier. Exemplary liquid carriers comprise polar organic solvents such as heavy aromatic naptha and xylene.

While this particular invention has been described with respect to particular embodiments thereof, it is apparent that numerous other forms and modifications of this invention will be obvious to those skilled in the art. The appended claims and this invention generally should be construed to cover all such obvious forms and modifications which are within the true spirit and scope of the present invention.

What is claimed is:

1. A method for inhibiting the polymerization of an activated vinyl monomer comprising contacting said monomer with an effective polymerization inhibiting amount of an aliphatic nitroso compound represented by the general formula
R—N═O (I)
wherein R represents an unsubstituted or substituted alkyl group.

2. A method as recited in claim 1 wherein said activated vinyl monomer is a member or members chosen from the group consisting of styrene, acrylic acid, acrylates, methacrylic acid, methacrylates, acrylamides, vinyl chloride, vinyl fluoride, and vinyl acetate and said activated vinyl monomer is heated to a temperature of 80° C. or higher.

3. A method as recited in claim 2 wherein said activated vinyl monomer is styrene.

4. A method as recited in claim 1 further comprising contacting said activated vinyl monomer with a member or members selected from the group consisting of (II) and (III), wherein (II) is a compound represented by the formula 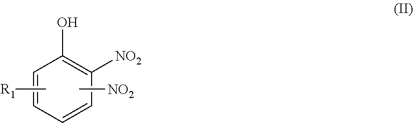
wherein R₁ is H or C₁-C₁₂ alkyl, and wherein (III) is represented by the formula 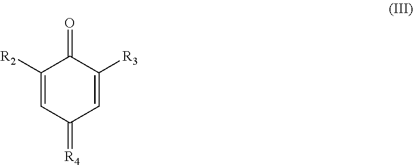
wherein R₂ and R₃ are independently H, C₁ to C₁₈ alkyl, C₅ to C₁₂ cycloalkyl; or C₇ to C₁₅ phenylalkyl, and R₄ is aryl, or aryl substituted with C₁ to C₆ alkyl, alkoxy, or hydroxy, nitro, amino, carboxy or mixtures thereof.

5. A method as recited in claim 3 wherein said aliphatic nitroso compound is 2-nitroso-2-methylpropane.

6. A method as recited in claim 3 wherein said aliphatic nitroso compound is 2-nitroso-2,4,4-trimethylpentane.

7. A method as recited in claim 4 wherein said activated vinyl monomer is an aromatic vinyl monomer.

8. A method as recited in claim 7 wherein said aromatic vinyl monomer is styrene, (I) is 2-nitroso-2-methylpropane and (II) is present and wherein (II) is 4,6-dinitro-o-cresol, 2,6-dinitro-p-cresol or 2-sec-butyl-4,6-dinitrophenol.

9. A method as recited in claim 8 wherein (II) is 2-sec-butyl-4,6-dinitrophenol.

10. A method as recited in claim 7 wherein said aromatic vinyl monomer is styrene, (I) is 2-nitroso-2 methylpropane and (III) is present and wherein (III) is 2,6-di-tertbutyl-4-benzylidene-cyclohexa-2,5 dien-1-one.

11. Activated vinyl monomer antipolymerization composition comprising a liquid carrier and dissolved or dispersed therein an aliphatic nitroso compound represented by the general formula
R—N═O (I)
wherein R represents an unsubstituted or substituted C₁-C₁₀ alkyl group and a member or members selected from the group consisting of (II) and (III) wherein (II) is a compound represented by the formula 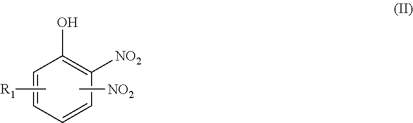
wherein R₁ is H or C₁-C₁₂ alkyl, and wherein (III) is represented by the formula 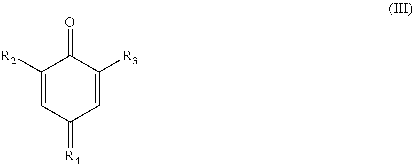
wherein R₂ and R₃ are independently H, C₁ to C₁₈ alkyl, C₅ to C₁₂ cycloalkyl; or C₇ to C₁₅ phenylalkyl, and R₄ is aryl, or aryl substituted with C₁ to C₆ alkyl, alkoxy, or hydroxy, nitro, amino, carboxy or mixtures thereof.

12. A composition as recited in claim 11 wherein (I) is present in a weight ratio of about 10-1 part (I) to about 1-10 parts of the combination of (II) and/or (III).

13. A composition as recited in claim 12 wherein said activated vinyl monomer is styrene.

14. A composition as recited in claim 13 wherein said aliphatic nitroso compound is 2-nitroso-2 methylpropane.

15. A composition as recited in claim 13 wherein said aliphatic nitroso compound is 2-nitroso-2,4,4-trimethylpentane.

16. A composition as recited in claim 14 wherein (II) is present and comprises 4,6-dinitro-o-cresol, 2,6-dinitro-p-cresol or 2-sec-butyl-4,6-dinitrophenol.

17. A composition as recited in claim 16 wherein (II) is 2-sec-butyl-4,6-dinitrophenol.

18. A composition as recited in claim 14 wherein (III) is present and comprises 2,6,-di-tertbutyl-4-benzylidene-cyclohexa-2,5 diene-1-one.
